(19) United States
(12) Patent Application Publication
(10) Pub. No.: US 2007/0224251 A1
(43) Pub. Date: Sep. 27, 2007

(54) Hemostatic material

(76) Inventors: Masao Tanihara, Kyoto-shi (JP); Hisao Kinoshita, Ikoma-shi (JP); Takayuki Imamura, Kikuchi-shi (JP); Chikateru Nozaki, Kikuchi-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/385,750

(22) Filed: Mar. 22, 2006

(51) Int. Cl. A61K 38/48; A61L 15/00

(52) U.S. Cl. 424/445; 424/94.64

(57) ABSTRACT

The present invention provides a hemostatic material which is excellent in hemostatic property, biodegradability and bioabsorbability, uniformity and stability of the quality, as well as reduces a risk of contamination with a pathogenic organism derived from an animal. The hemostatic material comprises a thrombin and a synthetic polypeptide capable of forming a triple helical structure. The polypeptide may show a peak of the molecular weight in the range from 5×10⁴ to 100×10⁴ in the molecular weight distribution. The polypeptide may contain at least a peptide unit represented by the formula: -Pro-X-Gly- (in the formula, X represents Pro or Hyp). The thrombin may be a recombinant. In the hemostatic material, the proportion of the thrombin may be about 0.1 to 500 units (U) relative to 1 mg of the polypeptide. The hemostatic material may further comprise a binder component having biodegradability and bioabsorbability. The hemostatic material may be formed on a substrate.

### FIELD OF THE INVENTION

The present invention relates to a novel hemostatic material which is excellent in a hemostatic effect and has an excellent bioaffinity or biocompatibility.

### BACKGROUND OF THE INVENTION

Hitherto, as a stanching method using a hemostatic material, has been adopted a method which comprises spraying or applying a hemostatic material such as an oxycellulose, a collagen, a gelatin, a calcium alginate, a thrombin, or a fibrin adhesive over a bleeding site. These hemostatic materials have been used in the form of a powder, a liquid, a fiber, a cloth or fabric (a nonwoven fabric), a film, a sponge, or others.

Japanese Patent Application Laid-Open No. 255830/1995 (JP-7-255830A) discloses a bioabsorbable surgical hemostatic material which comprises a cloth made of a neutralized oxycellulose containing 0.5 to 4.0% by weight of calcium. Japanese Patent Application Laid-Open No. 26578/2003 (JP-2003-26578A) discloses a hemostatic material comprising a maleate of a deacetylated chitin. Japanese Patent Application Laid-Open No. 369874/2002 (JP-2002-369874A) discloses a hemostatic material comprising a water-soluble fiber assembly such as a carboxymethylcellulose. Japanese Patent Application Laid-Open No. 60341/2002 (JP-2002-60341A) discloses that a hemostatic material containing a calcium salt of a nucleic acid as a main component can be applied even to an excessive bleeding site and accelerates spontaneous recovery of a damaged blood vessel without any problem of antigenicity. Japanese Patent Application Laid-Open No. 322614/1999 (JP-11-322614A) discloses a wound hemostatic material containing a carboxymethylcellulose and having a facilitatory effect for cell adhesion. Japanese Patent Application Laid-Open No. 169653/1997 (JP-9-169653A) discloses a chitin hemostatic agent which comprises a chitin fiber having an orientation degree of 50 to 98%. Japanese Patent Application Laid-Open No. 103479/1997 (JP-9-103479A) discloses a medical material (e.g., a bioadhesive, and a hemostatic material) in which a gelatin is crosslinked by a succinimidated polyglutamic acid. Japanese Patent Application Laid-Open No. 118157/1995 (JP-7-118157A) discloses a hemostatic material which is obtained by polymerization of D, L-lactide and a polyethylene glycol, wherein the molar ratio of ethylene oxide unit and lactic acid unit in the material is 52:48 to 30:70, and the molecular weight of the material is 7800 to 15000.

Japanese Patent Application Laid-Open No. 2971/1997 (JP-9-2971A) discloses a stable tissue adhesive containing an activator or proactivator of a prothrombin, and a prothrombin of less than 5 unit/gfibrinogen. Japanese Patent Application Laid-Open No. 35193/1996 (JP-8-35193A) discloses a process for producing a nonwoven sheet of a collagen fiber, which comprises discharging an acidic solution of a soluble collagen in a salt aqueous solution to give a collagen fiber, cutting the fiber, dispersing the cut fiber into a solvent and making a paper from the fiber. This document also discloses that the obtained nonwoven sheet is useful as a hemostatic material which can be promptly and effectively applied to a wound site. Moreover, Japanese Patent Publication No. 34830/1986 (JP-61-34830B) discloses a wound agglutination material which comprises a collagen carrier partially or wholly coated with a mixture of a fibrinogen component and a thrombin component. This document discloses a naturally occurring collagen or a chemically modified (or denatured) collagen as the collagen, and discloses that one derived from an animal or human can be used as the thrombin component.

However, according to these conventional hemostatic materials, it is difficult to stop bleeding effectively, particularly in the case of a rapid excessive loss of blood or an excessive bleeding. In particular, even in the case of spraying a powder or liquid hemostatic material directly to a bleeding site, a hemostatic component is easy to be carried away by the bloodstream. Moreover, in the conventional hemostatic materials, although temporary stypticity is recognized on some level, these hemostatic materials have low biodegradability and bioabsorbability, or contain a large amount of cytotoxic substance. Therefore, depending on the species of the hemostatic materials, it is sometimes necessary to remove the hemostatic material after blood stanching, and thereby there is a possibility of re-bleeding.

On the other hand, in biomaterial applications such as a hemostatic material, it is often the case that a raw material derived from an animal (e.g., bovine, and horse) is used. However, although the use of an animal-derived raw material can enhance biodegradability and bioabsorbability, there is a risk of contamination with a pathogen. Accordingly, the quality of the material is irregular. For example, a causative substance of bovine spongiform encephalopathy or sheep tremor is an infectious protein called as prion, and the infectious protein is considered as one of causes of human Creutzfeldt-Jakob disease infection. Prion is a protein, and it is indicated that prion is hard to deactivate with a conventional pasteurization or sterilization method, further that prion is infectious over species (Nature Review, Vol. 2, pp. 118 to 126, 2001).

Incidentally, in International Publication pamphlet No. 03/004641, the inventors of the present invention disclose a production process of a recombinant thrombin by genetic recombination technique.

### SUMMARY OF THE INVENTION

It is therefore an object of the present invention to provide a hemostatic material which is excellent in hemostatic property (or stypticity), is high in bioaffinity and biocompatibility, and has the same quality and an excellent stability.

It is another object of the present invention to provide a hemostatic material with a low risk of an infection (or a transmission) by a pathogenic organism (or a causative factor) or an undesirable side effect in the case of using a thrombin derived from human plasma or a recombinant thrombin as a thrombin (particularly, a recombinant thrombin).

It is still another object of the present invention to provide a hemostatic material which is degradable and absorbable in a living body.

It is further object of the present invention to provide a hemostatic material which can be formed into various shapes (or forms), and effectively stops bleeding as usage.

The inventors of the present invention made intensive studies to achieve the above objects and finally found that combination use of a chemically synthesized polypeptide having a triple helical structure and a thrombin ensures an excellent hemostatic property (or stypticity), and high bioaffinity and biocompatibility. The present invention was accomplished based on the above findings.

That is, the hemostatic material of the present invention comprises a thrombin, and a synthetic polypeptide capable of forming a triple helical structure. The polypeptide may show a peak of the molecular weight in the range from 5×10⁴ to 100×10⁴ in the molecular weight distribution. The polypeptide may contain at least a peptide unit represented by the formula: -Pro-X-Gly-(wherein X represents Pro or Hyp). The thrombin may be a recombinant. In the hemostatic material, the proportion of the thrombin may be about 0.1 to 500 units (U) relative to 1 mg of the polypeptide. The hemostatic material may further comprise a binder component having biodegradability and bioabsorbability (e.g., a polysaccharide or a derivative thereof, a peptide, and a biodegradable and bioabsorbable polyester). The proportion (weight ratio) of the binder component relative to the total amount of the thrombin and the polypeptide may be about 0.01/99.99 to 95/5. The hemostatic material may be formed on a substrate (or abase).

Further, the present invention includes a method for treating a wound site (e.g., a wound site of a human being), which comprises applying a hemostatic material to the wound site, wherein the hemostatic material contains a thrombin, and a synthetic polypeptide capable of forming a triple helical structure.

Incidentally, in the present invention, amino acid residues are abbreviated to the following condensation codes.
Ala: L-alanine residue
Arg: L-arginine residue
Asn: L-asparagine residue
Asp: L-aspartic acid residue
Cys: L-cysteine residue
Gin: L-glutamine residue
Glu: L-glutamic acid residue
Gly: glycin residue
His: L-histidine residue
Hyp: L-hydroxyproline residue
Ile: L-isoleucine residue
Leu: L-leucine residue
Lys: L-lysine residue
Met: L-methionine residue
Phe: L-phenylalanine residue
Pro: L-proline residue
Sar: sarcosine residue
Ser: L-serine residue
Thr: L-threonine residue
Trp: L-tryptophan residue
Tyr: L-tyrosine residue
Val: L-valine residue

Moreover, in this specification, amino acid sequences of peptide chains are represented in accordance with the conventional expression that N-terminus and C-terminus in an amino acid residue are drawn at the left and the right sides, respectively.

### DETAILED DESCRIPTION OF THE INVENTION

The hemostatic material of the present invention comprises a thrombin, and a synthetic polypeptide capable of forming a triple helical structure.

[Thrombin]

The thrombin is not particularly limited to a specific one as long as the thrombin has a blood coagulation action, and for example, there may be used a thrombin obtained by acting a calcium salt and thromboplastin on a prothrombin extracted from human or non-human animal plasma and purified. The use of a human thrombin can inhibit infection by pathogenic organisms derived from a non-human animal.

Moreover, the thrombin may include a recombinant thrombin obtained by genetic recombination technique, for example, a recombinant thrombin which is produced in a host such as Escherichia coli, a yeast, an insect cell, or an animal cell by using thrombin gene or prothrombin gene derived from human being or a non-human animal, and others. Such a recombinant thrombin has a uniform quality and can be stably provided. In addition, the recombinant thrombin can remarkably lower the risk of infection by a pathogenic organism derived from animal plasma.

For example, the recombinant prethrombin may be highly expressed by amplifying a prethrombin 2 gene by using a human prothrombin gene as a template, inserting the prethrombin gene to a high expression vector for, e.g., an animal cell (e.g., a cell derived from chicken, hamster, mouse, or human) host to construct a plasmid, and introducing the obtained expression plasmid into an animal cell. Further, the thrombin may be given by activating thus obtained recombinant prethrombin as a substrate with the use of a recombinant ecarin. The resulting thrombin may be usually purified by chromatography, or others. The details of the process for producing a recombinant thrombin may be referred to International Publication pamphlet No. 03/004641 by the inventors of the present invention.

The thrombin may be used singly or in combination.

[Polypeptide]

The polypeptide contained in the hemostatic material is not particularly limited to a specific one as long as the polypeptide is a synthetic polypeptide capable of forming a triple helical structure. It is sufficient that the synthetic polypeptide has a triple helical structure in at least a part of the polypeptide. The synthetic polypeptide having a triple helical structure forms a collagen-like (or collagenous) structure.

The polypeptide may show a peak of the molecular weight in the range from, for example, about 1×10⁴ to 500×10⁴, preferably about 2×10⁴ to 300×10⁴, and more preferably 5×10⁴ to 100×10⁴ in the molecular weight distribution. Too small molecular weight tends to reduce the hemostatic effect due to too high solubility. On the other hand, in the case where the molecular weight is too large, there is a possibility that processability is deteriorated because of too low solubility. Incidentally, the molecular weight (or the peak of the molecular weight) of the polypeptide may be, for example, determined in terms of a globular protein by means of an aqueous gel permeation chromatography (GPC).

The synthetic polypeptide having a triple helical structure may include a polypeptide containing at least a peptide unit represented by the formula: -Pro-X-Gly- (in the formula, X represents Pro or Hyp), and others. The synthetic polypeptide containing the peptide unit indicates an extremely stable triple helical structure. Moreover, due to a collagen-like fibrous form, the synthetic polypeptide is excellent inprocessability (or workability), and in addition, a shaped article formed from the polypeptide is excellent in strength.

The synthetic polypeptide may be a polypeptide (Pro-X-Gly)ₙ which comprises only the peptide unit: -Pro-X-Gly- (wherein, “n” denotes an integer of 1 to 20,000), or may be a polypeptide comprising the peptide unit: Pro-X-Gly- and other amino acid residue(s).

In the above-mentioned formula, the coefficient “n” may be preferably an integer of about 2 to 20,000 (e.g., about 10 to 10,000), more preferably an integer of about 30 to 10,000 (e.g., about 50 to 7,500), and particularly an integer of about 100 to 5,000 (e.g., about 150 to 4,000).

The polypeptide comprising only the polypeptide unit may include (Pro-Pro-Gly)ₙ, (Pro-Hyp-Gly)ₙ, and in addition, a polypeptide having both of the units: -Pro-Pro-Gly- and -Pro-Hyp-Gly-, wherein the total repeating number of the both units is “n”. In the polypeptide having the both of the units: -Pro-Pro-Gly-and -Pro-Hyp-Gly-, the ratio of the unit (Pro-Pro-Gly)ₙ₁ relative to the unit (Pro-Hyp-Gly)ₙ₂[n1/n2] may be about 0.1/99.9 to 99.9/0.1, preferably about 0.5/99.5 to 90/10, and more preferably about 1/99 to 80/20 (e.g., about 5/95 to 60/40). Incidentally, the numbers “n”, “n1” and “n2”represent the repeating numbers of the units (Pro-X-Gly)ₙ, (Pro-Pro-Gly)ₙ₁ and (Pro-Hyp-Gly)ₙ₂, respectively, and “n1” plus “n2” is “n”.

Other amino acid residue may include Ala, Arg, Asn, Asp, Cys, Gln, Glu, Gly, His, Hyp, Ile, Leu, Lys, Met, Phe, Pro, Sar, Ser, Thr, Trp, Tyr, Val, and others. These amino acid residues may be used singly or in combination.

Moreover, the polypeptide may have a dicarboxylic acid residue (e.g., a residue of an aliphatic dicarboxylic acid such as an alkanedicarboxylic acid), a diamine residue (e.g., an aliphatic diamine residue) and/or a lactam residue, and others within a range in which hemostatic property, or biodegradability and bioabsorbability is not inhibited.

These polypeptides may be used singly or in combination.

Moreover, the polypeptide may be a physiologically or pharmacologically acceptable salt, and for example, may be a salt with a salifiable compound such as an inorganic acid (e.g., a hydrochloric acid, a sulfuric acid, and a phosphoric acid), an organic acid (e.g., acetic acid, trifluoroacetic acid, lactic acid, tartaric acid, maleic acid, fumaric acid, oxalic acid, malic acid, citric acid, oleic acid, and palmitic acid), a metal (e.g., an alkali metal such as sodium or potassium, an alkaline earth metal such as calcium, and aluminum), or an organic base (e.g., trimethylamine, triethylamine, t-butylamine, benzylamine, diethanolamine, dicyclohexylamine, and arginine). These salifiable compounds may be used singly or in combination. These salts may be obtained by a conventional salt-forming reaction.

The polypeptide shows positive Cotton effect at a wavelength in range of 220 to 230 nm and negative Cotton effect at a wavelength in range of 195 to 205 nm in circular dichroism spectra. At least one part (that is, a part or all) of the polypeptide of the present invention is, accordingly, capable of forming a triple helical structure, and the polypeptide forms a collagenous (collagen-like) polypeptide. Incidentally, Cotton effect means a phenomenon caused by difference between an absorption coefficient relative to a right circularly polarized light and that relative to a left at a specific wavelength in an optical rotatory substance. Therefore, the formation of a triple helical structure in the polypeptide can be usually proved by measuring circular dichroism spectra for a solution of the polypeptide. Incidentally, regarding circular dichroism spectra, it has been reported that a naturally-occurring collagen and peptide chain forming a triple helical structure distinctively shows positive Cotton effect at a wavelength in range of 220 to 230 nm and negative Cotton effect at a wavelength in range of 195 to 205 nm (J. Mol. Biol., Vol. 63 pp. 85 to 99, 1972).

These polypeptides are capable of forming a collagen tissue (or a collagenous tissue or collagen-like tissue). The polypeptide chains forming the above-mentioned triple helical structure can self-assemble to form a fibril having a length of several nanometers to several tens nanometers. Further, these fibrils can be arranged to form a fiber structure having a length of several nanometers to several tens nanometers. These can be observed by a transmission electron microscope, a scanning electron microscope, or an atomic force microscope.

The polypeptide is, different from a collagen derived from mammals, free from a risk of an infection of a pathogenic organism or a transmission of a causative factor [for example, a protein converted into a pathological protein (e.g., abnormal prion)], and has a high safety. Moreover, the polypeptide is capable of forming a collagen-like polypeptide, and is also excellent in cytophilicity or biocompatibility.

The hemostatic material of the present invention is not particularly limited to a specific one as far as the material comprises the thrombin and the polypeptide. The hemostatic material may be used in the form of a liquid (e.g., a solution or a suspension), a non-liquid [for example, a particulate, a fiber, and a shaped article such as a two-dimensional shape (e.g., a woven fabric, a non-woven fabric, a film, and a sheet) or a three-dimensional shape (e.g., a sponge)].

Since the polypeptide can be easily formed into a desired shape due to having a high film-forming property or formability, the polypeptide may be suitably used for forming a hemostatic material containing the thrombin and the polypeptide. Moreover, forming or film-forming may be conducted by using the thrombin and the polypeptide, and if necessary a binder component (or a film-forming component) having biocompatibility (particular, biodegradability and bioabsorbability).

The binder component may include a polysaccharide or a derivative thereof [for example, a locust bean gum, a guar gum, a tragacanth gum, an alginic acid or a salt thereof (e.g., a sodium alginate), a propylene glycol alginate, a pectin, a starch, an amylose, an amylopectin, an agarose, an agar, a chitin, a chitosan, a carageenin, a hyaluronic acid, a chondroitin compound (e.g., a chondroitin sulfate, a sodium chondroitin sulfate, and a chondroitin heparin), a dextran, and a cellulose or a derivative thereof (e.g., a cellulose, a methylcellulose, an ethylcellulose, a carboxymethylcellulose, or a salt thereof, a cellulose ether such as a hydroxyethylcellulose, a hydroxypropylcellulose, or a hydroxypropylmethylcellulose, and a cellulose ester such as a cellulose acetate)], a peptide compound (e.g., a polypeptide such as a polylysine, a polyglutamine, or a polyglutamic acid; and a protein such as a gelatin, a casein, or an albumin), and a polyester-series resin [e.g., a biodegradable and bioabsorbable polyester such as a homo-or copolymer of a hydroxycarboxylic acid such as glycolic acid, lactic acid, 3-hydroxybutyric acid, 4-hydroxybutyric acid, or 3-hydroxypropionic acid (e.g., a lactic acid-glycolic acid copolyester); and a copolyester of a hydroxycarboxylic acid, and propionic acid, and a lactone (e.g., butyrolactone, and caprolactone)]. These binder components may be used singly or in combination. In the case of using such a binder component, the strength or formability of the hemostatic material can be improved, or the water-absorbing property thereof can be adjusted.

The hemostatic material of the present invention may contain other additive(s), for example, other hemostatic component (e.g., a fibrinogen, and an oxycellulose), a cell adhesion protein (e.g., a fibronectin, a vitronectin, and a laminin), an antibacterial agent, a preservative, and a salt (e.g., a physiologically acceptable salt).

The hemostatic material may be a hemostatic material in which the thrombin is applied to a substrate formed by the polypeptide by coating or impregnation. The shape or configuration of the substrate formed by the polypeptide is not particularly limited to a specific one, and may be in the form of a particulate (e.g., a particulate having a size of about 1 to 300 μm), a one-dimensional shape (e.g., a fiber or filament form, a linear form, and a rod form), a two-dimensional shape (e.g., a film (or sheet) or a plate form), and a three-dimensional shape (e.g., a tube form). Further, the polypeptide substrate may be a non-porous body, or a porous body. The polypeptide substrate may include, for example, (1) a fibrous polypeptide obtained by extruding a solution or suspension of a polypeptide into a solution containing a high concentration of a salt or a polypeptide-insoluble solvent through a nozzle, and coagulating the extruded matter, (2) a non-woven fabric obtained from the fibrous polypeptide with the use of a wet or dry paper production process, (3) a sponge-like porous body obtained by leaving an aqueous solution or suspension of a polypeptide as it is, or if necessary with crosslinking the polypeptide by adding a crosslinking agent, to prepare a gel matter, and lyophilizing the gel matter, and (4) a porous body obtained by stirring and foaming an aqueous solution or suspension of a polypeptide and drying the solution or suspension. Moreover, if necessary, the polypeptide substrate may be crosslinked by a physiologically acceptable crosslinking agent, for example, a dialdehyde compound such as glyoxal, glutaraldehyde or succinaldehyde, a dextrandialdehyde, and an aldehyde starch.

Further, the hemostatic material may be formed on a substrate by applying a hemostatic component at least containing a thrombin and a polypeptide to the substrate. Such a substrate usually has bioaffinity, and biocompatibility in many cases, and may include, for example, a polysaccharide or a derivative thereof (e.g., a polysaccharide such as an alginate, a chitin, a chitosan, a hyaluronic acid, a polygalactosamine, a curdlan, a pullulan, axanthan, or adextran, a cellulose or a derivative thereof as described in the paragraph of the above-mentioned binder component, a protein (e.g., a gelatin, a casein, and an albumin), a polypeptide (e.g., a polylysine, a polyglutamine, and a polyglutamic acid), a vinyl alcohol-series resin (e.g., a polyvinyl alcohol-series resin such as a polyvinyl alcohol, and an ethylene-vinyl alcohol copolymer), a polyvinyl pyrrolidone-series resin, an acrylic resin (e.g., a (meth)acrylic acid-series resin such as a poly(meth)acrylic acid, or a (meth)acrylic acid copolymer), a halogen-containing resin (e.g., a fluorine-containing resin such as a polytetrafluoroethylene, and a vinyl chloride-series resin such as a polyvinyl chloride), a polyurethane-series resin, a silicone-series resin, a polyester-series resin as described in the paragraph of the above-mentioned binder component, and a polyamide-series resin (e.g., a nylon 6, and a nylon 66). The substrates may be used singly or in combination.

The substrate may have a non-biodegradability or bioerodability. It is advantageous that the substrate has degradability and absorbability in a living body. Such a biodegradable substrate may comprise a biodegradable resin. The biodegradable resin may include various resins, for example, the polysaccharide or the derivative thereof, and the polyester-series resin. Incidentally, the substrate may be a composite substrate using not less than two kinds of materials.

The shape or configuration of the substrate is not particularly limited to a specific one, and according to purposes, may be the same as the shape or configuration mentioned in the paragraph of the polypeptide substrate. Moreover, the substrate may be a non-porous body, or a porous body (for example, a particulate porous body, a cellulose fiber paper, a two-dimensional porous body such as a non-woven fabric or a woven fabric, and a three-dimensional porous body having a cylindrical form). If necessary, the substrate may be surface-treated with a finishing (or surface-treating) agent (e.g., a physiologically acceptable finishing agent).

### Proportion of Each Component

The proportion of the thrombin in the hemostatic material is not particularly limited to a specific one as long as the material has a hemostatic action, and may be, for example, selected from the range of about 0.1 to 500 units, preferably about 0.2 to 300 units, and more preferably about 0.3 to 200 units, relative to 1 g of the hemostatic material. Too low proportion of the thrombin tends to make the hemostatic effect insufficient. In the case where the proportion of the thrombin is too high, there is a possibility that the thrombin cannot act efficiently.

Moreover, depending on the shape of the hemostatic material, for example, in a liquid hemostatic material, the proportion (concentration) of the thrombin may be, e.g., about 3 to 200 units/mL, preferably 5 to 150 units/mL, and more preferably 10 to 100 units/mL. In a non-liquid hemostatic material, the proportion of the thrombin may be, for example, about 0.1 to 30 units, preferably about 0.3 to 10 units, and more preferably about 0.5 to 5 units, relative to 1 g of the hemostatic material. In a hemostatic material formed on a substrate, the proportion of the thrombin may be, for example, about 10 to 500 units/cm², preferably about 20 to 300 units/cm², and more preferably about 30 to 200 units/cm², relative to 1 cm² of the surface area at which the hemostatic component is applied to the substrate.

Further, the proportion of the thrombin may be, for example, about 0.1 to 500 units, preferably 0.1 to 300 units (e.g., about 0.1 to 100 units), and more preferably about 0.5 to 50 units (e.g., about 1 to 20 units) relative to 1 mg of the polypeptide.

The proportion of the polypeptide is not particularly limited to a specific one as long as the polypeptide promotes the hemostatic action of the thrombin, enhances adhesiveness between the hemostatic material and a tissue, and maintains strength and flexibility of a shaped article formed from the hemostatic material. For example, the proportion of the polypeptide may be about 0.01 to 95% by weight, preferably about 0.05 to 90% by weight, and more preferably about 0.1 to 85% by weight relative to the whole hemostatic material (hemostatic component). Moreover, according to the shape of the hemostatic material, for example, in a liquid hemostatic material, the proportion of the polypeptide may be, e.g., within the range of about 0.01 to 20% by weight, preferably about 0.01 to 10% by weight, and more preferably about 0.05 to 5% by weigh relative to the whole hemostatic material. In a non-liquid hemostatic material, the proportion of the polypeptide may be, for example, about 1 to 95% by weight, preferably about 5 to 90% by weight, and more preferably about 5 to 80% by weight relative to the whole hemostatic material (hemostatic component).

The proportion of the binder component may be within a range at which the binder component exhibits desired strength or water absorbing property without inhibiting the hemostatic action of the hemostatic material. For example, the proportion (weight ratio) of the binder component relative to the total amount of the thrombin and the polypeptide may be selected from the range of about 0.01/99.99 to 95/5, preferably about 0.05/99.95 to 90/10, and about more preferably 0.1/99.9 to 85/15. Moreover, depending on the shape of the hemostatic material, for example, in a liquid hemostatic material, the proportion (weight ratio) may be, for example, about 0.01/99.99 to 20/80, preferably about 0.01/99.99 to 10/90, and about more preferably 0.05/99.95to 5/95. In a non-liquid hemostatic material, the proportion (weight ratio) maybe, for example, about 1/99 to 90/10, preferably about 2/98 to 70/30, and more preferably about 2/98 to 60/40.

### Production Process of Hemostatic Material

The hemostatic material of the present invention may be produced by a conventional method. For example, a liquid hemostatic material may be prepared by dissolving or dispersing a hemostatic component at least containing the thrombin and the polypeptide in water, a physiological saline, an organic solvent (e.g., a mild organic solvent such as propanol or glycerin), or a mixed solvent thereof.

A particulate hemostatic material may be, for example, prepared by pulverizing the polypeptide or spray-drying a solution or suspension of the polypeptide to give a particulate polypeptide, and mixing thus obtained particulate polypeptide and a particulate thrombin; and

spray-drying a solution or suspension containing the thrombin and the polypeptide. Further, a sheet- or film-formed hemostatic material may be obtained by flow-casting a solution or suspension containing the hemostatic component and if necessary the binder component on a strippable support (e.g., a glass plate, a fluorine-containing resin (a polytetrafluoroethylene) sheet, and a fluorine-containing resin-coated vat), and drying the solution or suspension. A sponge-like hemostatic material may be given by leaving a solution (or suspension) or gel matter containing the hemostatic component as it is, or if necessary with adding a cross linking agent, or lyophilizing the solution (or suspension) or gel matter. A fibrous hemostatic material may be, for example, obtained by injecting a solution or suspension containing the hemostatic component to a coagulation bath such as an aqueous solution containing a high concentration of a salt (e.g., sodium sulfate), or ethanol through a nozzle or other means for fiber forming. Thus obtained fibrous hemostatic material may be shaped (or molded) by a conventional method to prepare a woven fabric- or non-woven fabric-formed hemostatic material.

Moreover, a hemostatic material formed on a substrate may be produced by applying the hemostatic component at least containing the thrombin and the polypeptide to at least the surface of the substrate. For example, the hemostatic material comprising the substrate having a surface coated by the hemostatic component may be obtained by coating or spraying (or impregnating) the surface of the substrate with a solution or suspension of the hemostatic component, and then drying the resulting matter. Further, a porous substrate (e.g., a non-woven fabric) may be impregnated with a solution or suspension of the hemostatic component to give a hemostatic material holding (or carrying) the hemostatic component. Incidentally, the hemostatic component may be applied to a site to be adapted to a living body in the substrate (a site in contact with not only body tissues but also body fluid or blood). In a particulate or one-dimensional shaped substrate, the hemostatic component may be applied to the whole substrate. In a two-dimensional shaped substrate, the hemostatic component may be applied to at least one surface of the substrate. In a three-dimensional shaped substrate, the hemostatic component may be applied to a site to be adapted to a living body (e.g., the whole area, the internal surface, and the external surface).

Incidentally, a hemostatic material in which the thrombin is applied to a polypeptide substrate may be also prepared by the same matter as described above, for example, by applying a component containing at least the thrombin to a polypeptide substrate with the use of coating, spraying (or dispersion), or impregnation.

According to the present invention, the combination of a thrombin and a polypeptide having a collagen-like structure ensures excellent hemostatic property (or stypticity), and high bioaffinity and biocompatibility, as well as uniform quality and excellent stability. Moreover, in the case of using a human plasma thrombin or a recombinant thrombin (particularly, a recombinant thrombin) as a thrombin, there is little possibility of an infection (or a transmission) by a pathogenic organism (or a causative factor) or an undesirable side effect, and is high safety. Further, such a thrombin is biodegradable and bioabsorbable. In addition, the hemostatic material of the present invention is high in formability (or moldability) and can be formed or molded into various shapes. Therefore, the hemostatic material can effectively stop bleeding depending on applications.

The hemostatic material of the present invention is useful for effectively treating a wound site of an animal (e.g., a damage or injury of a skin or organ) by applying the hemostatic material to the wound site. Such an animal may include human beings, and nonhuman animals (e.g., reptiles, birds, fish, and mammals). Examples of the mammals may include monkeys, sheep, bovines, horses, dogs, cats, rabbits, rats, and mice.

The application of the hemostatic material to the wound site is not particularly limited to a specific one, and may be suitably set or determined depending on the shape of the hemostatic material, the position-or condition of the wound site, and others. For example, in the sheet- or film-formed hemostatic material (as well as the sponge-like hemostatic material, the woven fabric- or non-woven fabric-formed hemostatic material), the wound site and the hemostatic material may be adhered to each other by covering part or whole of the wound site with the hemostatic material, and oppressing the wound site. Moreover, if necessary, the hemostatic material may be fixed on (or around) the wound site with the use of an adhesive or suture thread which may be biodegradable or bioabsorbable. Further, the particulate hemostatic material may be applied to the wound site by spraying. Incidentally, after applying the hemostatic material to the wound site, if necessary, the wound site and the hemostatic material may be protected with a cover sheet, a bandage, or others.

Incidentally, in the case of applying the hemostatic material having biodegradability and bioabsorbability to a wound site of a living body, the hemostatic material can effectively stop bleeding, as well as it is unnecessary to remove the hemostatic material from the wound site after the bleeding stopped. Such a hemostatic material, therefore, is effective for not only a wound site of an outer skin but also a wound of an internal organ. In particular, due to excellent hemostatic property (or stypticity) and tissue adhesiveness, the hemostatic material of the present invention is also effective in stopping of excessive bleeding following a damage or an operation of an internal organ (e.g., lung, and liver).

### EXAMPLES

The following examples are intended to describe this invention in further detail and should by no means be interpreted as defining the scope of the invention.

### Example 1

A peptide (1 g) represented by the formula: Pro-Hyp-Gly (manufactured by Peptide Institute, Inc.) was dissolved in 20 mL of 10 mM phosphate buffer solution (pH 7.4). To the peptide solution was added 473 mg of 1-hydroxybenzotriazole and 3.35 g of 1-ethyl-3-(3-dimethylaminopropyl)-carbodiimide hydrochloride. The mixture was stirred at 4° C. for 2 hours, and the stirring was continued at 20° C. for 46 hours. The reaction solution was dialyzed against MilliQ (ultrapure water) for 48 hours.

The resulting solution after dialysis was diluted 50-fold with water, and the diluted solution was subjected to a gel-permeation chromatography (AKTA purifier system, manufactured by Amarsham Bioscience K.K., column: Superdex 200HR10/30, flow rate: 0.5mL/min., eluent: 10 mM phosphate buffer (pH 7.4) containing 150 mM NaCl). As a result, the peak of the molecular weight of the polypeptide was recognized in the range from 100000 to 600000 in the molecular weight distribution.

Moreover, the resulting solution after dialysis was diluted 100-fold with water, the diluted solution was subjected to a circular dichroism spectrum measurement, and positive Cotton effect was observed at a wavelength of 225 nm and negative Cotton effect at a wavelength of 198 nm. The results confirmed that the polypeptide formed a triple helical structure.

A working curve was created based on absorbance of a peptide represented by the formula:

H-(Pro-Hyp-Gly)₁₀-OH (Sequence ID No. 1) (manufactured by Peptide Institute, Inc.) at 215 nm, and was used to determine the concentration of the resulting chemosynthetic polypeptide forming a triple helical structure as about 20 mg/mL.

A polyglycolic acid non-woven fabric “NEOVEIL” (manufactured by Gunze Limited) cut into 3 cm around was impregnated with about 700 U of a recombinant thrombin (manufactured by Juridical Foundation The Chemo-Sero-Therapeutic Research Institute: referred to International Publication No. 03/004641 pamphlet), and lyophilized. Then, the lyophilized fabric was impregnated with 0.5 mL of the chemosynthetic polypeptide forming a triple helical structure which was diluted to a concentration of about 20 mg/mL, and lyophilized to give a non-woven fabric hemostatic material.

### Comparative Example 1

A non-woven fabric hemostatic material was obtained in the same manner as Example 1 except for using 0.5 mL of a pig Type III collagen (manufactured by Nitta Gelatin Inc.) instead of the chemosynthetic polypeptide forming a triple helical structure.

### Comparative Example 2

A non-woven fabric hemostatic material was obtained in the same manner as Example 1 except that the polyglycolic acid non-woven fabric was impregnated with 0.5 mL of the diluted chemosynthetic polypeptide forming a triple helical structure and lyophilized without application to the recombinant thrombin.

### Example 2

2.5 mL of an aqueous solution of sodium alginate (manufactured by Kimika Corporation, 99 mPa·s) having a concentration of 1% by weight, 2.5 mL of the chemosynthetic polypeptide (20 mg/mL) forming a triple helical structure obtained by Example 1, and 0.34 mL of a recombinant thrombin (manufactured by Juridical Foundation The Chemo-Sero-Therapeutic Research Institute: referred to International Publication No. 03/004641 pamphlet) having a concentration of 2000 U/mL were mixed. The mixture was flow-cast into a Teflon (registered trademark) tray having inner dimensions of 3 cm around, and then air-dried at a room temperature to give a sheet hemostatic material.

### Example 3

2.5 mL of an aqueous solution of sodium alginate (manufactured by Kimika Corporation, 99 mPa·s) having a concentration of 1% by weight, 2.5 mL of the chemosynthetic polypeptide (20 mg/mL) forming a triple helical structure obtained by Example 1, and 0.34 mL of a recombinant thrombin (manufactured by Juridical Foundation The Chemo-Sero-Therapeutic Research Institute: referred to International Publication No. 03/004641 pamphlet) having a concentration of 2000 U/mL were mixed. The mixture was flow-cast into a Teflon (registered trademark) tray having inner dimensions of 3 cm around, and then lyophilized to give a sponge hemostatic material.

### Example 4

The chemosynthetic polypeptide forming a triple helical structure obtained by Example 1 (having a concentration of 20 mg/mL) was diluted with MilliQ to a concentration of 15 mg/mL. The diluted matter (2.25 mL) was flow-cast into a polyethylene tray having inner dimensions of 3 cm around, and air-dried at a room temperature in a clean bench to obtain a sheet. The chemosynthetic polypeptide (20 mg/mL) forming a triple helical structure obtained by Example 1 was diluted with MilliQ to a concentration of 10 mg/mL, and 4.5 mL of the diluted solution was flow-cast on the obtained sheet, and immediately lyophilized to give a sponge layer of the synthetic polypeptide. Thereafter, the obtained sponge layer was impregnated with 0.34 mL of a recombinant thrombin (manufactured by Juridical Foundation The Chemo-Sero-Therapeutic Research Institute: referred to International Publication No. 03/004641 pamphlet) having a concentration of 2000U/mL, and lyophilized again to obtain a hemostatic material composed of two layers, that is, a sheet layer and the sponge layer.

### Test Example

Japanese white rabbit liver was exposed, and an epidermis thereof was exfoliated to create a circular avulsed wound having a diameter of 12 mm. Then, with each of the hemostatic materials obtained by Examples 1 to 4 and Comparative Examples 1 and 2, the wound was coated, and oppressed for 1 minute. A filter paper was allowed to absorb the blood leaked out from the hemostatic material until the bleeding was stopped, and the total amount of the bleeding was determined based on the weight of the filter paper.

The results were proved the total amount of the bleeding in the hemostatic material obtained by Example 1 was 0.316 g (the average of three measurements (n =3)), the amount in the hemostatic material obtained by the Example 2 was 0.521 g (the average of three measurements (n =3)), the amount in the hemostatic material obtained by Example 3 was 0.296 g (the average of three measurements (n =3)), and the amount in the hemostatic material obtained by Example 4 was 0.346 g (the average of three measurements (n =3)). On the other hand, the total amount of the bleeding in the hemostatic material obtained by Comparative Example 1 was 1.895 g (the average of three measurements (n =3)), and the amount in the hemostatic material obtained by Comparative Example 2 was 1.270 g (the average of three measurements (n =3)).

It is apparent from Test Example that the hemostatic materials obtained by Examples 1 to 4 clearly reduce the total amount of the bleeding compared with Comparative Examples 1 and 2, and are excellent in a hemostatic effect.

1. A hemostatic material containing a thrombin, and a synthetic polypeptide capable of forming a triple helical structure,
wherein the synthetic polypeptide is at least one member selected from the group consisting of
(i) a polypeptide containing a unit represented by (Pro-Pro-Gly)ₙ,
(ii) a polypeptide containing a unit represented by (Pro-Hyp-Gly)ₙ, and
(iii) a polypeptide containing a unit represented by (Pro-Pro-Gly)ₙ₁, and a unit represented by (Pro-Hyp-Gly)ₙ₂,
wherein, in the polypeptides (i) to (iii), each of “n”, “n1” and “n2” represents a repeating number of each unit, n1/n2 is 0.1/99.9 to 99.9/0.1, “n1” plus “n2” is “n”, and “n” is an integer of 2 to 20,000.

2. A hemostatic material according to claim 1, wherein the polypeptide shows a peak of the molecular weight in the range from 5×10⁴ to 100×10⁴ in the molecular weight distribution.

3-4. (canceled)

5. A hemostatic material according to claim 1, wherein the thrombin is a recombinant.

6. A hemostatic material according to claim 1, wherein the proportion of the thrombin is 0.1 to 500 units relative to 1 mg of the polypeptide.

7. A hemostatic material according to claim 1, which further comprises a binder component having biodegradability and bioabsorbability.

8. A hemostatic material according to claim 7, wherein the binder component comprises at least one member selected from the group consisting of a polysaccharide, a peptide, and a biodegradable and bioabsorbable polyester.

9. A hemostatic material according to claim 7, wherein the proportion (weight ratio) of the binder component relative to the total amount of the thrombin and the polypeptide is 0.01/99.99 to 95/5.

10. A hemostatic material according to claim 1, which is formed on a substrate.

11. A method for treating a wound site, which comprises applying a hemostatic material to said wound site, wherein the hemostatic material contains a thrombin, and a synthetic polypeptide capable of forming a triple helical structure according to claim 1.

12. A method according to claim 11, wherein the wound site is a wound site of a human being.

13. A hemostatic material according to claim 1, wherein “n” is an integer of 10 to 20,000.


SEQUENCE LISTING (1 sequence)

SEQ ID NO: 1 - protein, 30 aa, Artificial Sequence
  Inventor Tanihara, Masao Inventor Kinoshita, Hisao Inventor Imamura, Takayuki Inventor Nozaki, Chikateru
  Description of Artificial Sequencepeptide
  MISC_FEATURE (1)..(30): Xaa is defined as 4Hyp
PXGPXGPXGPXGPXGPXGPXGPXGPXGPXG